(19) United States
(12) Patent Application Publication
(10) Pub. No.: US 2012/0304983 A1
(43) Pub. Date: Dec. 6, 2012

(54) NASAL SPRAY

(76) Inventors: Jochen Ganz, Uster (CH); Josefine Lacour, Dielsdorf (CH); Alexander Panos, Wettswil (CH); Stefan Stahl, Berikon (CH)

(21) Appl. No.: 13/519,641

(22) PCT Filed: Dec. 24, 2010
(86) PCT No.: PCT/EP2010/070724

§ 371 Date: Jun. 28, 2012

(30) Foreign Application Priority Data
Dec. 28, 2009 (CH) 2000/09

(51) Int. Cl. A61M 11/00; A24F 47/00; B65D 83/00

(52) U.S. Cl. 128/200.14; 222/251; 222/372; 222/1; 131/270

(57) ABSTRACT

A container for holding and dispensing a liquid and/or dissolved active substance, for example a pharmaceutical or cosmetic active substance, has at least one dispensing organ (5) for dispensing, preferably in a portioned manner, the one active substance. Provision is also made for at least two holding chambers (9, 11) separated from one another, at least one of which contains the one active substance and at least one of which contains an at least largely active-substance-free liquid and/or a further active substance differing from the one active substance, wherein the dispensing organ is connected to the at least one chamber containing the active substance for dispensing purposes.

### RELATED APPLICATION

This application is a U.S. national phase application of International Application No. PCT/EP2010/070724 filed Dec. 24, 2010 with claiming priority of Switzerland patent application no. 2000/09 filed Dec. 28, 2009.

### TECHNICAL FIELD

The present invention relates to a container for holding and dispensing a liquid and/or dissolved active substance, more particularly a pharmaceutical or cosmetic active substance, the container having at least one dispensing organ, to an applicator comprising a container and to a method for changing the concentration of a pharmaceutical or cosmetic active substance in the case of metered dispensing by means of an aforementioned container.

### BACKGROUND AND SUMMARY

These days, many people repeatedly use decongestant nasal sprays when they have a common cold. Within 5-7 days, said sprays lead to a bodily counterregulation and dependence. This problem leads to there being patients with a nasal-spray dependency stretching over many years (partly without even noticing this) and, conversely, many patients reject such a nasal spray from the outset due to fear of becoming dependent. This problem is addressed in various studies.

There is an analogous problem in cosmetics; for example, excessively long-term use of so-called anti-dandruff shampoos often leads to side-effects.

A solution to the stated problem is proposed in U.S. Pat. No. 5,988,870, according to which a titration system is provided for periodic manual dilution using a saline solution in a nasal spray. The dilution in this case is intentional and so, occasionally, it is possible to determine a worsening of the effect of the nasal spray in the user for purely psychological reasons.

It is therefore an object of the present invention to propose a solution according to which the side-effects suffered by the patient can be reduced, for example when using a nasal spray or when using an anti-dandruff shampoo.

According to the invention, a container for holding and dispensing at least one liquid and/or dissolved active substance, particularly a pharmaceutical or cosmetic active substance, having at least one dispensing organ for dispensing, is proposed accordingly, and also a method for changing the concentration of an active substance, more particularly a pharmaceutical or cosmetic active substance, when the latter is dispensed to a patient and/or user.

A container is proposed for holding and dispensing a liquid and/or dissolved active substance, for example a pharmaceutical or cosmetic active substance, having at least one dispensing organ for dispensing, preferably in a portioned manner, the one active substance, and also having at least two holding chambers separated from one another, at least one containing the at least one active substance and at least one further one containing an at least largely active-substance-free liquid and/or at least one further active substance differing from the one active substance. The dispensing organ is connected to the at least one chamber containing the one active substance for dispensing purposes.

According to an embodiment variant, the at least one chamber containing the one active substance and the further chamber are connected to one another via a valve such that active-substance-free liquid and/or the further active substance can be driven into the chamber containing the one active substance.

It is also proposed for the dispensing organ to be operatively connected to actuation means such that air can be driven into the further chamber by the actuation means in the case of at least some of the actuations of the dispensing organ and as result of this active-substance-free liquid and/or the further active substance can be driven into the chamber containing the one active substance.

According to in turn a further embodiment variant, it is proposed that means for feeding air are provided both on the chamber containing the one active substance and on the further chamber and that the means for feeding air are operatively connected to the dispensing organ such that it is possible to set manually or automatically whether air is fed into the chamber containing the active substance or into the further chamber.

According to in turn a further embodiment variant, it is proposed that provision is made for a plurality of chambers containing the one active substance with different concentrations of active substance and the dispensing organ is, when actuated, initially connected to the chamber having the highest concentration of the one active substance, subsequently connected to the chamber having the second-highest concentration of the one active substance, etc. until, lastly, liquid is dispensed from that chamber that is largely free from the one active substance or only has the active-substance-free liquid and/or a further active substance.

The container proposed according to the invention is particularly suited for a nasal spray in which the problem mentioned at the outset is reduced by successively decreasing the concentration of active substance. The functional principle of the nasal spray proposed according to the invention lies in the fact that a mechanism in the pump spray automatically dilutes the dispensed liquid more and more in terms of active substance after a certain lead-in phase. The lead-in phase can have a duration of 0 days, a few days or else take a relatively long time, depending on the severity of the disorder to be treated. The mechanism can also be implemented in a semi-automatic manner.

Additionally, it is possible for the further chamber to contain at least one further active substance differing from the one active substance in addition to the active-substance-free liquid provided for the dilution. By way of example, this further active substance can be an active substance that promotes wound healing.

It is self-evident that the container proposed according to the invention is also suitable for e.g. cosmetic active substances, where the dependence of applying an e.g. cosmetic liquid should be successively reduced. The use of an anti-dandruff shampoo is mentioned as an example.

Furthermore, a method is also proposed for changing the concentration of an active substance, more particularly a pharmaceutical or cosmetic active substance, during metered dispensing, wherein initially one or more metered amounts are dispensed from a first holding chamber, containing the one active substance, of a container of the type described above by means of a dispensing organ, an amount of a largely active-substance-free liquid and/or of a further active substance which differs from the one active substance, contained in a second, further chamber, is transferred into the first chamber after dispensing a first amount or after dispensing a plurality of amounts of the one active substance in order to reduce the concentration of the one active substance.

### BRIEF DESCRIPTION OF DRAWINGS

The invention will now be explained in more detail in an exemplary manner and with reference to the attached figures.

Here:

FIG. 1 schematically shows a section through a first embodiment variant of a nasal spray according to the invention;

FIG. 2 schematically shows a section through a further embodiment variant of a nasal spray according to the invention;

FIG. 3 schematically shows a section through a further embodiment variant of a nasal spray according to the invention;

FIG. 4 shows a diagram of a possible concentration profile when an active substance is dispensed by means of a nasal spray according to the invention;

FIG. 5 shows a diagram of the concentration profile of the one active substance and of an additional further active substance during the diluting procedure;

FIG. 6 schematically shows a section through a detail of the nasal spray as per FIG. 1, illustrating the suctioning of the active ingredient by the suction pump;

FIG. 7 shows the same detail from FIG. 6, but schematically illustrating the suctioning of dilution solution by the dispensing pump; and

FIGS. 8 and 9 schematically show the air intake in the region of the dispensing pump for feeding the chamber with active substance or the chamber containing the dilution liquid.

### DETAILED DESCRIPTION OF EMBODIMENTS

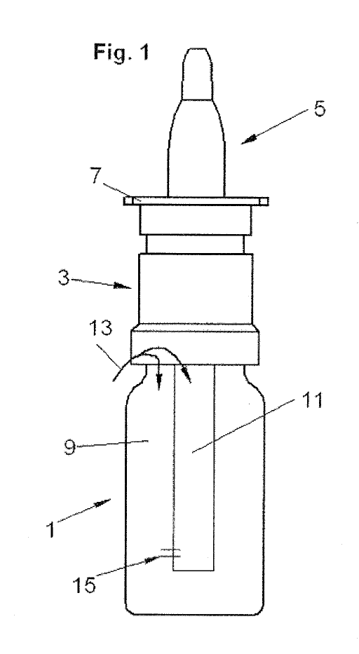

FIG. 1 schematically shows a section through a first embodiment variant of a nasal spray 1 according to the invention. The vessel 1 is subdivided into two chambers 9 and 11. The dissolved one active ingredient is situated in the inner chamber 11. The dissolved one active ingredient preferably is a pharmaceutical mixture for alleviating symptoms of the common cold. In the outer one 9, there is a solvent with or without additional active ingredient. Air is suctioned in to replace the pumped-out volume every time the dispensing organ 5 is pumped; the actuation of the latter can be effected by means of a collar 7. During a lead-in phase, air is suctioned in from the outside, as indicated schematically by reference sign 13, and routed into the inner chamber 11. The dilution is now initiated by the air now being routed into the outer chamber 9 instead of the inner one, said outer chamber being connected to the inner chamber 11 via a valve 15. Whether air is routed to the inner or the outer chamber can be regulated by means of an air-inlet mechanism, which is arranged in the cover part 3. This air-inlet mechanism can be actuated during each pump action, wherein the air is driven or suctioned into the outer chamber after the aforementioned lead-in time and hence solvent, optionally containing an additional active ingredient, is pumped from the outer into the inner chamber, as a result of which the concentration of the one active ingredient in the inner chamber 11 is successively reduced. That is to say the active substance concentration in the inner chamber is successively reduced with each pump action.

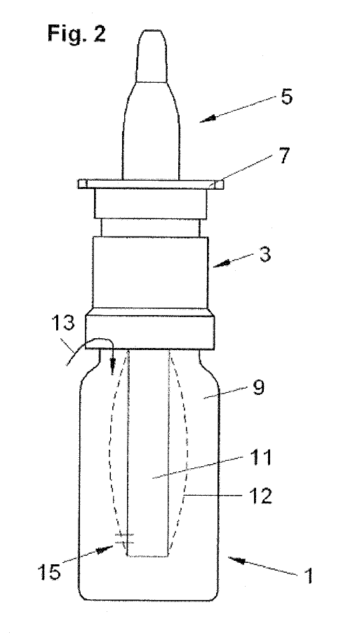

FIG. 2 shows a further embodiment variant which is analogous to the one from FIG. 1, which the difference that the inner chamber 11 has a flexible wall 12 and this leads to the option of routing the air into the outer chamber 9 from the outset. The lead-in phase ends as soon as the flexible chamber has reached its minimum volume; a valve 15 is opened thereafter and this in turn transports the liquid, optionally with additional active ingredient, into the inner chamber 11.

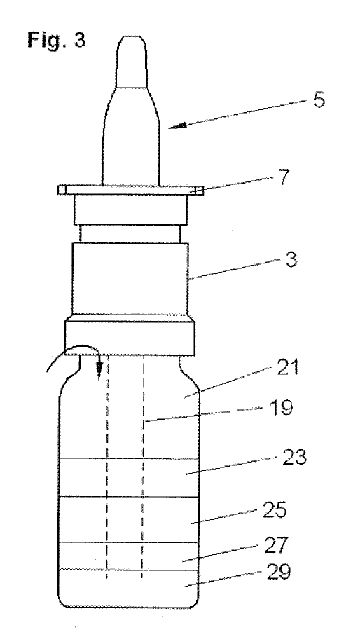

FIG. 3 in turn illustrates a further embodiment variant of a nasal spray according to the invention, wherein the vessel is subdivided into a plurality of chambers 21 to with different active ingredient concentrations. A mechanism which, for example, is once again arranged in the cover part 13, guides the suction tube 19 step by step from one chamber into the next one having the next lower concentration, after a specific number of pump actions. Ultimately, liquid, optionally without an active ingredient, possibly supplemented with an additional active ingredient, is in turn routed to the user by the dispensing pump.

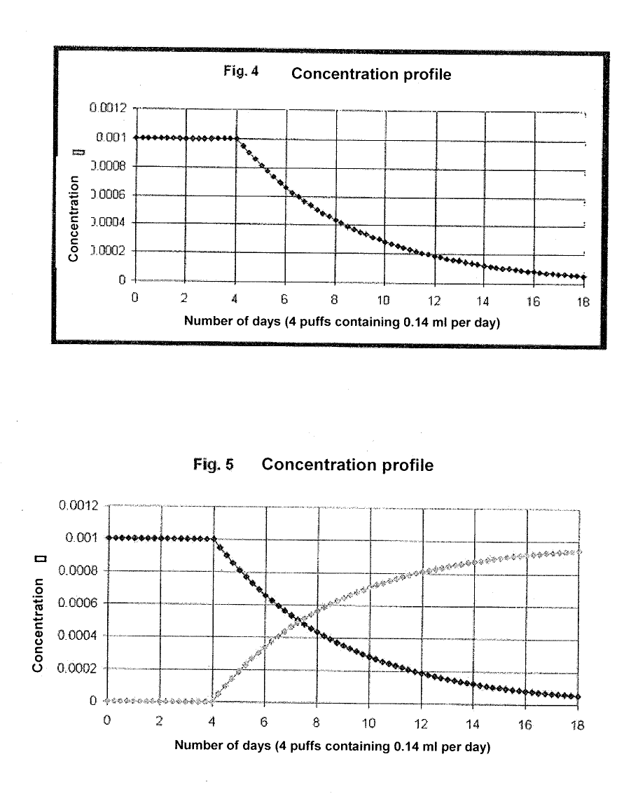

FIG. 4 schematically shows a diagram of a possible concentration profile when an active substance is dispensed by means of a nasal spray according to the invention. According to the illustrated example, the active ingredient concentration is not reduced during the first 4 days and only after the fourth day is active-ingredient-free liquid added to the active-ingredient-containing chamber, leading to a reduction in the concentration. After 18 days the dispensed spray is practically active-ingredient free. This provides a very effective contribution in respect of the problem mentioned at the outset.

Naturally, the concentration profile in FIG. 4 is an example, with, according to the invention, it also being possible to select a different concentration profile. The applicator according to the invention provides for total freedom, i.e. in respect of the substance selection, the application duration, when the diluting is initiated, the degree of dilution, etc. Moreover, it is possible to add an additional active ingredient to the diluting liquid. As a variant, reference is made in an exemplary fashion to an active ingredient with a wound-healing promoting effect, which is successively added to the first active ingredient, said first active ingredient for example having a decongesting effect. A corresponding concentration profile is illustrated in the diagram of FIG. 5, wherein, first, the concentration of the first active ingredient successively decreases and, secondly, the concentration of the wound-healing substance increases with increasing application use.

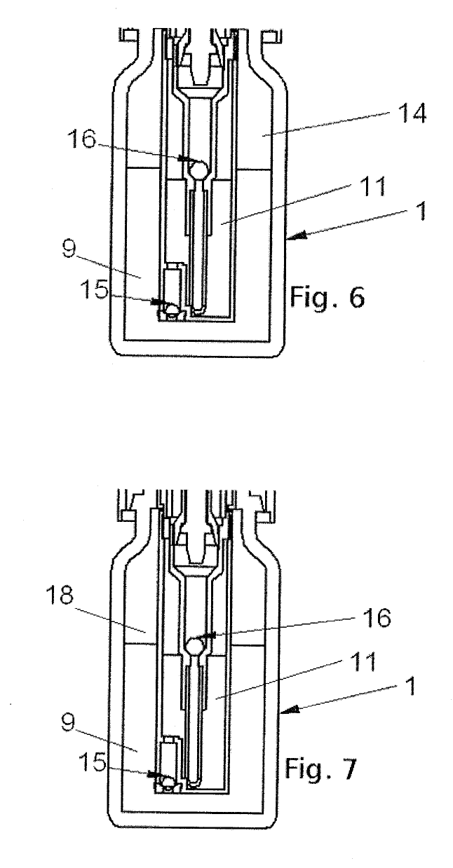

FIGS. 6 and 7 illustrate, as a detail, a section through the base part of a container of the nasal spray from FIG. 1. According to the illustration from FIG. 6, air is supplied to the chamber 11 containing the one active substance via channels 14 when the dispensing pump is actuated, as a result of which the active-ingredient-containing liquid is suctioned and dispensed by the dispensing pump 16 via the valve 16.

After a certain amount of time, such as, for example, the 4 days illustrated in FIG. 4, the air is now supplied via channels 18 to that chamber which in this case contains largely active-ingredient-free liquid, as a result of which dilution solution is suctioned into the chamber 11 via the valve 15 when the dispensing pump is actuated. As a result, there is a dilution in the chamber 11, as a result of which a diluted solution is suctioned and dispensed by the dispensing pump via the valve 16 after a certain amount of time.

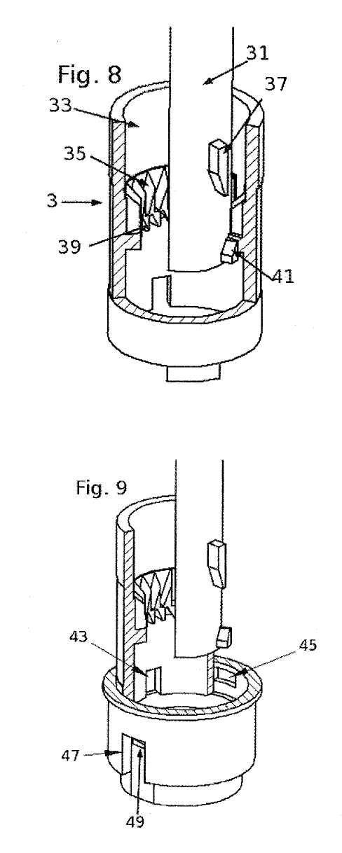

FIGS. 8 and 9 finally schematically illustrate a setting mechanism, by means of which the air supply into the individual chambers can be set. The mechanism, which is operatively connected to the dispensing pump, is preferably a rotating mechanism which carries out a small rotation during each pump action. When the dispensing organ is actuated, i.e. when a release tube 31 is lifted through the collar 7, appropriately beveled tooth-like contours 35 and 39 on the inner wall of the collar 3 are used to rotate the latter slightly by engaging into corresponding beveled projections 37 and 41 on the outer wall of the tube 31. This rotation renders possible to control the air supply to the chambers, as described with reference to FIGS. 1 to 3 and/or 6 and 7. By way of example, this can be brought about by means of air supply openings 45 and 47 in an outer ring, which openings, for the purposes of air passage, communicate with corresponding openings 43 and 49 in the ring-like wall 33 of the cover part 3. As a result of this rotation, the inlet valves 45 and 47 can be controlled for the air such that air is supplied to that chamber which contains the active substance in an initial phase and, in a subsequent phase, to that chamber which contains the largely active-ingredient-free liquid optionally with an additional active ingredient.

It is self-evident that the examples illustrated in FIGS. 1 to 9 are merely possible embodiment variants to aid the understanding of the present invention. The solution proposed according to the invention lends itself in particular to nasal sprays. Naturally, the proposed solution can be applied to any type of liquid in which e.g. the concentration of an active substance should be changed after a certain amount of time. This is because this problem occurs not only in nasal sprays but can naturally also occur in other active substances, more particularly in pharmaceutical and cosmetic active substances. In particular, the idea according to the invention can be applied in the case of sprays where a pump mechanism should be actuated.

As a matter of form, reference is made in the field of cosmetics to the use of so-called anti-dandruff shampoos, where it is likewise desirable if the concentration of dandruff-inhibiting active substances is reduced after a certain amount of time and, alternatively, an increase in lipid-replenishing active substances is desirable.

The mechanism for regulating the air supply illustrated with reference to FIGS. 8 and 9 can also be solved in a different manner and should merely illustrate a possible embodiment variant. Optionally, it is also possible to dispense with such a mechanism entirely if the active substance is diluted when dispensed from the outset.

1. Container for holding and dispensing at least one liquid and/or dissolved active substance, the container comprising at least one dispensing organ for dispensing the active substance, at least two holding chambers separated from one another, at least one of the chambers containing the at least one active substance and at least one further chamber of the chambers containing at least one of an at least largely active-substance-free liquid and a further active substance differing from the at least one active substance, wherein the dispensing organ is connected to the at least one chamber containing the active substance for dispensing purposes.

2. Container according to claim 1, further comprising a valve connecting the at least one chamber containing the at least one active substance and the further chamber such that liquid in the further chamber can be driven into the chamber containing the one active substance.

3. Container according to claim 1, wherein the at least one dispensing organ is operatively connected to actuation means for driving air into the further chamber in the case of at least some of the actuations of the dispensing organ and as result of this liquid can be driven into the chamber containing the at least one active substance.

4. Container according to claim 1, further comprising means for feeding air provided both on the chamber containing the one active ingredient and on the further chamber, and means for setting the air fed by said means for feeding air, said means for setting being operatively connected to the dispensing organ and settable as to whether air is fed into one chamber or the other chamber.

5. Container according to claim 3, wherein the means for setting the air fed includes a rotating mechanism operable by the dispensing organ to carry out a small rotation with every actuation of the dispensing organ, as a result of which, after a certain rotation, the air supply into the chamber containing the one active substance is interrupted and the air supply into the further chamber is opened.

6. Container according to claim 1, further comprising a plurality of chambers with different concentrations of the one active substance and wherein the dispensing organ is initially connected to that chamber for dispensing liquid containing active substance which has the highest concentration of the one active substance, liquid being removed from that chamber which has the second-highest concentration of the one active substance after this chamber was emptied until finally that chamber is emptied which has the active-substance-free liquid and/or the highest concentration of the further active substance.

7. Container of claim 1, wherein the active substance is a nasal spray.

8. Method for changing the concentration of at least one active substance selected from a group consisting of a pharmaceutical active substance and a cosmetic active substance, when dispensed in a metered manner, the method comprising:
initially dispensing one or more metered amounts from at least one first holding chamber, containing the at least one active substance, of a container by means of a dispensing organ,
transferring into the at least one first holding chamber as a diluting liquid an amount of at least one of a largely active-substance-free liquid and at least one further active substance which differs from the at least one active substance and is contained in a further chamber to reduce the concentration of the at least one active substance in the at least one first chamber.

9. Method according to claim 8, wherein the diluting liquid from the further chamber is transferred in portions into the at least one first holding chamber containing the at least one active substance, reducing the concentration of the at least one active substance in each case by at least 0.001% and at most 90%.

10. Method according to claim 8, including repeatedly dispensing a metered amount from the at least one first holding chamber, and in each dispensing transferring an amount of the diluting liquid from the further chamber into the at least one first holding chamber to further reduce the concentration of the at least one active substance in the at least one first holding chamber.

11. The method of claim 8, wherein the at least one active substance is a nasal spray.

12. The method of claim 8, wherein the at least one active substance is a decongestant nasal spray.

13. The method of claim 8, wherein the at least one active substance is an anti-dandruff shampoo.

14. The method of claim 8, wherein the container is an oral applicator.

15. The method of claim 8, wherein the container is an applicator for giving up smoking.

16. The method of claim 8, wherein the container is a trans-dermal applicator.

17. The method of claim 8, wherein the active substance is an anti-aging means.
